(19) United States
(12) Patent Application Publication
(10) Pub. No.: US 2008/0275129 A1
(43) Pub. Date: Nov. 6, 2008

(54) Methods and Pharmaceutical Compositions for the Treatment of Neurological Damage

(75) Inventors: Torbjorn Lundstedt, Uppsala (SE); Elisabeth Seifert, Uppsala (SE); Per Lek, Uppsala (SE); Arne Boman, Uppsala (SE); Anna Skottner, Ekero (SE)

Correspondence Address: BACON & THOMAS, PLLC, 625 SLATERS LANE, FOURTH FLOOR, ALEXANDRIA, VA, 22314-1176, US

(73) Assignee: Acure Pharma AB, Uppsala (SE)

(21) Appl. No.: 11/885,594

(22) PCT Filed: Mar. 6, 2006
(86) PCT No.: PCT/GB2006/000774

§ 371 Date: May 29, 2008

(30) Foreign Application Priority Data
Mar. 4, 2005 (SE) 0500500-4

(51) Int. Cl. A61K 31/155; A61P 25/00

(52) U.S. Cl. 514/632

(57) ABSTRACT

The invention relates to a method of treating neurological damage, preferably spinal chord injury, said treatment comprising the topical administration of guanabenz. The invention also relates to the use of guanabenz for the preparation of a medicament for the treatment of neurological damage, preferably spinal chord injury, wherein said treatment comprises the topical administration of guanabenz to the site of said neurological damage. The invention is also directed to pharmaceutical compositions comprising guanabenz wherein said compositions are in the form of an aqueous gel or non-aqueous ointment.

The present invention relates to the treatment of neurological damage, particularly the treatment of spinal cord injury, by the topical administration of guanabenz. The invention also relates to a pharmaceutical composition for topical administration that comprises guanabenz. More particularly the invention relates to compositions in the form of an aqueous gel or a non-aqueous ointment for the treatment of neurological damage by topical administration.

Spinal cord injury (SCI) is a serious clinical situation causing disability and suffering of patients due to progressive cell death, oedema and necrosis. The primary injury induces a series of secondary events that determine the extent of cell damage within the spinal cord. The most common types of SCI are contusion and compression, but laceration is also frequently reported.

Damage to motor nerve fibres results in paralysis while damage to sensory nerve fibres results in loss of sensation, e.g. touch, pressure and temperature. Other consequences are exaggerated reflexes, loss of bladder and bowel control, sexual dysfunction, impaired coughing reflexes, spasticity and reduced breathing capacity.

A number of large linear and cyclic peptides which show high specific binding to melanocortin (MC) receptors are known in the art. The agonistic and/or antagonistic properties of these peptides are also known. See WO 99/21571, for example.

Small molecules that bind to the MC receptors have been disclosed, for example, in WO 99/55679 and WO 99/64002. Hydroxyguanidines are discussed in WO 98/23267, and benzylideneamino guanidines in U.S. Pat. No. 4,060,640. Other examples of pharmacologically active guanidines are disclosed in U.S. Pat. No. 3,982,020 and GB 1223491. The use of guanidines in other application areas is known in the art and described in patents U.S. Pat. No. 3,896,332, DE 1165013, and U.S. Pat. No. 3,941,825, for example. Guanabenz is a compound that is well known in the art for use as an antihypertensive drug (US Pharmacopeia, 1999, The United States Pharmacopeial Convention, Inc, ISBN 1-889788-03-1). The neuroprotective effect of guanabenz has also been shown in a number of publications such as N. E. Naftchi, Int. J Devl. Neuroscience, 9 (2) 1991, 113-126; S. F. Morrison and Y. Z. Ge, J. Pharmacol. Exp. Ther. 273 (1) 1995, 380-385; and J. R. Jr Sotolongo et al, J. Urol. 146 (2) 1991, 426. The positive effect of aminoguanidines on spinal cord injury was reported in Sharma et al. Acta Neurochirurgica, Supplement (2003), 86, 399-405.

Drug delivery systems comprising hyaluronic acid and an active substance have been reported in EP-A-0224987. The compositions disclosed therein are said to be suitable for administration by injection, implantation or for topical application to the eye or skin.

We have surprisingly found that the topical application of guanabenz for the treatment of neurological damage, particularly spinal chord injury, is more effective at reducing the damage to the nerve or spinal chord that occurs after the primary injury than previously known methods of treatment.

We have also surprisingly found that pharmaceutical compositions comprising guanabenz wherein the composition is in the form of an aqueous gel or non-aqueous ointment have a particularly advantageous effect on the regeneration of nerves and tissue after neurological damage.

### STATEMENT OF INVENTION

The present invention relates to a method of treating neurological damage in a human or animal, said treatment comprising the topical administration of an effective amount of guanabenz to the site of neurological damage. The invention preferably relates to the treatment of spinal chord injury.

The invention also relates to the use of guanabenz for the preparation of a medicament for the treatment of neurological damage in a human or animal, wherein said treatment comprises the topical administration of an effective amount of said guanabenz to the site of said neurological damage.

The present invention is also directed to guanabenz for the topical treatment of neurological damage.

In addition, the invention is directed to a pharmaceutical composition comprising guanabenz water and a gel-forming agent.

In an alternative embodiment, the invention is directed to a pharmaceutical composition comprising guanabenz wherein the composition is in the form of a non-aqueous ointment.

The invention is also directed to the use of the compositions as defined above for the preparation of a medicament for the treatment of neurological damage in a human or animal, wherein said treatment comprises the topical application of an effective amount of said composition to the site of said neurological damage.

The invention also relates to compositions as defined above for use in therapy.

### BRIEF DESCRIPTION OF THE DRAWINGS

FIG. 1 shows the results of the Tarlov Score test.

FIG. 2 shows the results of the Capacity Angle test.

### DETAILED DESCRIPTION OF THE INVENTION

The present invention is directed to methods and compositions for the treatment of neurological damage. The invention is preferably directed to compositions and methods for the treatment of spinal chord injury.

The inventors have surprisingly found that the topical administration of guanabenz to a site of neurological damage results in improvements over the known methods of treating neurological damage using guanabenz. In particular, topical application of guanabenz is more effective at reducing damage to the nerve or spinal chord that occurs after the primary injury than previous known methods of treatment. It is postulated that topical application of guanabenz helps to reduce further cell damage that occurs after the primary injury by reducing oedema and/or apoptosis caused by the primary injury. Furthermore, the guanabenz starts to take effect more quickly when applied topically.

Since the guanabenz is applied topically, there is a decreased risk of systemic side effects.

In addition, the inventors have found that pharmaceutical gel or ointment compositions comprising guanabenz improve the treatment of neurological damage, particularly spinal chord injury, when the compositions are administered topically to the site of neurological damage. This is because the compositions of the invention are thought to allow controlled release of the guanabenz directly to the site of need (i.e. the site of neurological damage). In addition, the compositions of the invention comprise a biocompatible matrix and do not therefore produce any adverse reaction in the body.

The compositions of the invention provide guanabenz in a form that is manageable, easy to use, and in which guanabenz can be conveniently dispersed.

By “neurological damage” is meant nerve damage, preferably damage to the central nervous system.

By “the site of neurological damage” is meant the part of the nerve that is damaged by injury. In the spinal chord this can be defined by the discs at which the compression, laceration, or contusion, for example, has occurred.

By “topical administration” is meant local administration at or near the site of neurological damage, preferably administration directly on to the part of the nerve or spinal chord that has been damaged.

By “guanabenz” is meant [N-(2,6-dichlorophenyl)methyleneamino]guanidine, its tautomeric forms and pharmacologically acceptable derivatives and salts thereof.

By “gel-forming agent” is meant a substance that increases viscosity in order to produce a gel in an aqueous system.

By “hyaluronic acid” is meant hyaluronic acid, salts thereof such as the sodium salt, and derivatives thereof such as cross-linked derivatives as disclosed in EP-A-0224987, preferably the free acid or salts thereof.

### Pharmaceutical Compositions

Guanabenz used according to the present invention is provided in form that is suitable for topical administration to an area of neurological damage, particularly to an area of the spinal chord that has been damaged.

The guanabenz can be formulated in water, for example.

The pharmaceutical compositions of the present invention are preferably stabilised. For the purpose of this invention, the compositions can be stabilised using a stabiliser (e.g. a surfactant) or by controlling the conditions of the composition (e.g. by controlling the pH), or a combination of these factors. The stabilisation preferably prevents degradation of the guanabenz or collapse of the physical state of the composition. Variables that can affect the degradation include the solvent used, the composition pH, the presence of a buffer salt, age of the composition, exposure to light and the presence of other excipients or diluents. The compositions of the invention are preferably stabilised to the extent that, on storage at 25° C. in the dark for 21 days, less than 10% of the guanabenz degrades into other compounds. For some applications however, it is sufficient that less than 20% of the guanabenz degrades under these conditions. Conversely, other applications may require that less than 0.1% or even that less than 0.01% or 0.001% degradation occurs under these conditions. Degradation of guanabenz can be detected using known HPLC systems that are suitable for detecting benzylidineaminoguanidines.

The inventors have surprisingly found that pharmaceutical compositions in the form of an aqueous gel or non-aqueous ointment comprising guanabenz are particularly effective when administered topically to treat neurological damage, particularly spinal chord injury.

### Aqueous Gel Compositions

In a further embodiment, the invention is directed to a pharmaceutical composition comprising guanabenz wherein the composition is in the form of an aqueous gel. These compositions comprise guanabenz, a gel-forming agent and water.

Preferred gel-forming agents include glucosaminoglycans, such as, for example, hyaluronic acid (HYA) or cellulose derivatives such as, for example, hydroxyethyl cellulose (HYA) and carboxymethyl cellulose (CMC). A preferred gel-forming agent is hyaluronic acid. These gel-forming agents are commercially available.

Guanabenz is present in the aqueous gel compositions of the invention in various amounts, according to the particular needs of the recipient. Suitable amounts, as % w/w of the composition, may be from 0.0001-10, more preferably 0.001-1.0, desirably 0.01-0.2, and most desirably 0.06-0.1%, for example.

The amount of gel-forming agent in the compositions of the invention may vary. Suitable amounts, as % w/w of the composition, may be from 0.0001-20, such as 0.001-10, 0.01-10, 0.01-5, 0.1-5, 1-5 or 1-3, for example.

The aqueous gel compositions are preferably have pH values ranging from 3 to 11. A high degree of acidity or alkalinity is not desirable in formulations that are to be applied to the body. Preferably the pH ranges from 6 to 9, more preferably from 6.8 to 7.6. The compositions preferably comprise a pH buffer. A variety of buffers are suitable and would be well known to the skilled person. Sodium phosphate buffer and citrate-phosphate-borate buffer are suitable, for example.

The aqueous gel formulations preferably comprise one or more ionic surfactants. The presence of an ionic surfactant in an aqueous formulation of guanabenz improves the stability of the guanabenz in the formulation.

Suitable ionic surfactants include anionic surfactants, for example sodium lauryl sulphate (SDS), and cationic surfactants, for example cetrimide. SDS can be used in stable formulations at a neutral or near-neutral pH. If present, the amount of ionic surfactant in the composition may vary, but suitable amounts, as % w/w of the composition, may be from 0.0001-10, such as 0.001-5, 0.01-5, 0.01-2, 0.1-2 or 0.5-1.5.

Other pharmacologically acceptable components or excipients may also be present in the aqueous formulations.

The compositions preferably comprise excipients such as aromatic or aliphatic alcohols, preferably benzyl alcohol, ethanol or isopropanol. Benzyl alcohol is particularly preferred to assist in solubilising the guanabenz in the aqueous formulation. If present, the amount of alcohol may vary, but suitable amounts, as % w/w of the composition, may be from 0-10, such as 0.001-5, 0.01-5, 0.1-5 or 1-3.

The aqueous gels may be prepared by simply mixing the ingredients, though sonication may assist preparation. One possible method of preparation includes adding all the ingredients except the gel-forming agent to the vessel, sonicating this mixture for a few minutes, for example from one to five minutes, preferably around three minutes, to solubilise the guanabenz and the other solutes, adding the gel-forming agent, and manually mixing the resultant mixture.

Preferred aqueous gel compositions of the invention comprise guanabenz, hyaluronic acid, SDS, sodium phosphate buffer and optionally benzyl alcohol or ethanol.

### Non-Aqueous Ointments

An alternative embodiment of the present invention is directed to a pharmaceutical composition comprising guanabenz wherein the composition is in the form of anon-aqueous ointment.

We have found that dissolving or dispersing guanabenz in a non-aqueous system improves the guanabenz stability.

The amount of guanabenz present in the non-aqueous ointment compositions of the invention may be varied according to the particular needs of the recipient. Suitable amounts, as % w/w of the composition, may be from 0.0001-2, such as 0.0001-1, 0.001-0.5, 0.01-0.2 or 0.05-0.15, for example.

The compositions according to this embodiment preferably comprise an organic solvent and an ointment emulsifier.

Preferred ointment emulsifiers include commercially available emulsifiers such as elastomers, cyclomethicone, isopropyl myristate and cetomacrogol. Cetomacrogol is preferred.

Preferred organic solvents include aliphatic or aromatic alcohols, preferably ethanol, isopropanol or benzyl alcohol. If present the amount of alcohol may vary, but suitable amounts, as % w/w of the composition may be from 1-20, such as from 2-15 or 4-11.

Oleaginous solvents may also be added to improve the spreadability of the formulation. Suitable oleaginous solvents would be well know to the person skilled in the art and include Miglyol 812, for example. If present, the amount of oleaginous solvent may vary, but suitable amounts, as % w/w, of the composition, may be from 1-40, such as 10-30, 20-30 or 24-26, for example.

Other components such as diluents or excipients such as those well known to the person skilled in the art may be present in the non-aqueous ointment formulation.

One preferred ointment composition comprises guanabenz, ethanol, Miglyol 812 and cetomacrogol. A second preferred combination comprises guanabenz, isopropanol and an oily base comprising an elastomer, cyclomethicone and, optionally, isopropyl myristate. The oily base preferably comprises as % w/w of the oily base 79-90% elastomer, 10-21% cyclomethicone and 0-2% isopropyl myristate.

The term “non-aqueous ointment” does not imply that water is absolutely excluded—this would clearly be very difficult and unnecessary in most cases. A small amount of water may be present without adversely affecting the formulation. For example, the non-aqueous ointment may contain, as % w/w of the composition <5%, preferably <1%, <0.5%, <0.1% or <0.01% water.

The non-aqueous ointments may be prepared simply by mixing the ingredients. In order to ease this process, all the ingredients except the ointment emulsifier may be vortexed and/or sonicated for a short time, for example up to one minute, to solubilise the guanabenz and other solutes. The ointment emulsifier may be separately heated to 60° C. in a water bath, and then the mixture added to this heated component. The heated mixture may then be further mixed or homogenised.

### Treatment of Neurological Damage

Treatment of acute spinal chord injury generally involves surgery on the spinal chord. In particular, surgery would usually involve opening the spinal chord in order to release pressure and therefore reduce further damage caused by oedema.

Topical administration of guanabenz in accordance with the present invention therefore involves applying guanabenz to the part of the spinal chord that has been damaged, or close to the site of damage, when the spinal chord is exposed during surgery. The compositions of the invention can be applied to the exposed spinal chord or near to the exposed spinal chord using methods known to the skilled person, such as by syringe, for example. The compositions are preferably applied such that the area of neurological damage is covered by the composition.

Treatment is preferably started within a period of 15 hours after the injury has occurred.

The dose of guanabenz is preferably 0.1 to 10 mg/kg of body weight. Treatment preferably comprises administration of a single dose of guanabenz in a controlled release formulation, which will allow for the guanabenz to be released over a period of up to five days. Treatment may alternatively comprise multiple administrations of lower doses over a period of up to five days, however.

The gels and ointments of the present invention are able to release the active substance to the site of injury over a period of several hours or even days. This means that a single application of a gel or ointment composition of the invention is sufficient to provide treatment over an extended period. Thus, multiple intrusive surgical procedures can be avoided.

### EXAMPLES

The following examples are intended to illustrate but not limit the scope of the invention.

### ABBREVIATIONS

PB: 0.05 M solution of sodium phosphate buffer in water
CPB: 0.05 M solution of citrate-phosphate-borate buffer in water
BA: benzyl alcohol
EA: ethanol

### IPA: Isopropylalcohol

SDS: Sodium lauryl sulphate
HYA: Hyaluronic acid in the form of the free acid or the sodium salt

Guanabenz was obtained from Sigma-Aldrich. Hyaluronic acid (the free acid and the sodium salt) was obtained from Biochemica (Fluka).

The compositions shown in Table 1 were made by mixing the components together, as described above.

TABLE 1
Examples of preferred formulations

| Formulation | Components | (% w/w) |
|---|---|---|
| Aqueous Gel A | Guanabenz | 0.095 |
| | SDS | 1.0 |
| | HYA | 2.0 |
| | PB (pH 7.18) | 96.905 |
| Aqueous Gel A control | Guanabenz | 0 |
| | SDS | 1.0 |
| | HYA | 2.0 |
| | PB (pH 7.18) | 97.0 |
| Aqueous Gel B | Guanabenz | 10 |
| | SDS | 0 |
| | HYA | 2.0 |
| | PB (pH 7.18) | 97.34-97.35 |
| Aqueous Gel B control | Guanabenz | 0 |
| | SDS | 0.60 |
| | HYA | 2.0 |
| | PB (pH 7.18) | 97.4 |
| Aqueous Gel C | Guanabenz | 0.35 |
| | Cetrimide | 1.0 |
| | HYA | 2.0 |
| | CPB (pH 10.31) | 96.65 |
| Aqueous Gel C control | Guanabenz | 0 |
| | Cetrimide | 1.0 |
| | HYA | 2.0 |
| | CPB (pH 10.31) | 97.0 |
| Aqueous Gel F | Guanabenz | 0.01 |
| | SDS | 0.8 |
| | HYA | 1.5 |
| | BA | 3.0 |
| | PB (pH 7.5) | 94.64 |
| Aqueous gel F control | Guanabenz | 0 |
| | SDS | 0.8 |
| | HYA | 1.5 |
| | BA | 3.0 |
| | PB (pH 7.5) | 94.7 |
| Aqueous gel G | Guanabenz | 0.1 |
| | SDS | 0.8 |
| | HYA | 1.5 |
| | EA | 1.0 |
| | PB (pH 6.9) | 96.6 |
| Aqueous gel G control | Guanabenz | 0 |
| | SDS | 0.8 |
| | HYA | 1.5 |
| | BA | 1.0 |
| | PB (pH 6.9) | 96.7 |
| Aqueous gel H | Guanabenz | 0.06 |
| | SDS | 0 |
| | HYA | 0.1 |
| | BA | 3.0 |
| | PB (pH 6.9) | 94.24 |
| Aqueous gel H control | Guanabenz | 0 |
| | SDS | 1.2 |
| | HYA | 1.5 |
| | BA | 3.0 |
| | PB (pH 6.9) | 94.3 |
| Aqueous gel I | Guanabenz | 0.1 |
| | SDS | 0 |
| | HYA | 1.5 |
| | iPA | 1.0 |
| | PB (pH 7.5) | 97.4 |
| Aqueous gel I control | Guanabenz | 0 |
| | SDS | 1.2 |
| | HYA | 1.5 |
| | BA | 1.0 |
| | PB (pH 7.5) | 96.3 |
| Aqueous gel J | Guanabenz | 0.06 |
| | SDS | 0.8 |
| | HYA | 2.5 |
| | BA | 1.0 |
| | PB (pH 7.5) | 95.64 |
| Aqueous gel J control | Guanabenz | 0 |
| | SDS | 0.8 |
| | HYA | 2.5 |
| | BA | 1.0 |
| | PB (pH 7.5) | 95.64 |
| Aqueous gel K | Guanabenz | 1 |
| | SDS | 0 |
| | HYA | 1 |
| | EA | 3.0 |
| | PB (pH 6.9) | 95 |
| Aqueous gel K2 | Guanabenz | 1 |
| | SDS | 0 |
| | HYA | 0 |
| | EA | 3.0 |
| | PB (pH 6.9) | 96 |
| Aqueous gel L | Guanabenz | 0.06 |
| | SDS | 1.2 |
| | HYA | 2.5 |
| | iPA | 1.0 |
| | PB (pH 6.9) | 95.24 |
| Aqueous gel L control | Guanabenz | 0 |
| | SDS | 1.2 |
| | HYA | 2.5 |
| | BA | 1.0 |
| | PB (pH 6.9) | 95.3 |
| Aqueous gel M | Guanabenz | 0.1 |
| | SDS | 1.2 |
| | HYA | 2.5 |
| | EA | 3.0 |
| | PB (pH 7.5) | 93.2 |
| Aqueous gel M control | Guanabenz | 0 |
| | SDS | 1.2 |
| | HYA | 2.5 |
| | BA | 3.0 |
| | PB (pH 7.5) | 93.3 |
| Aqueous gel N | Guanabenz | 0.01 |
| | SDS | 1.0 |
| | HYA | 2.0 |
| | BA | 2.0 |
| | PB (pH 7.2) | 94.99 |
| Aqueous gel N control | Guanabenz | 0 |
| | SDS | 0.0 |
| | HYA | 1.0 |
| | EA | 4.0 |
| | PB (pH 7.2) | 95.0 |
| Aqueous gel O | Guanabenz | 0.06 |
| | SDS | 0.8 |
| | HYA | 0.5 |
| | BA | 3.0 |
| | PB (pH 7.5) | 95.64 |
| Aqueous gel O control | Guanabenz | 0 |
| | SDS | 0.8 |
| | HYA | 2.5 |
| | BA | 1.0 |
| | PB (pH 7.5) | 95.64 |
| Aqueous gel P | Guanabenz | 0.1 |
| | SDS | 0 |
| | HYA | 1.5 |
| | BA | 3.0 |
| | PB (pH 6.9) | 95.4 |
| Aqueous gel P control | Guanabenz | 0 |
| | SDS | 0.8 |
| | HYA | 2.5 |
| | iPA | 3.0 |
| | PB (pH 6.9) | 93.6 |
| Aqueous gel Q | Guanabenz | 1 |
| | SDS | 0 |
| | HYA | 1 |
| | BA | 1.0 |
| | PB (pH 6.9) | 97 |
| Aqueous gel Q control | Guanabenz | 0 |
| | SDS | 1.2 |
| | HYA | 2.5 |
| | BA | 1.0 |
| | PB (pH 6.9) | 95.3 |
| Aqueous gel R | Guanabenz | 0.1 |
| | SDS | 0 |
| | HYA | 1 |
| | EA | 3.0 |
| | PB (pH 7.5) | 95.9 |

### Effect of Guanabenz on Spinal Cord Injury

AP713 (Guanabenz), AP137 (reference compound: ME10501 see Sharma et al. Acta Neurochir (Wien), Suppl. 86: 399-405), and dexamethasone were topically administered to an area of spinal chord injury in rats. Dexamethosone is an anti-inflammatory compound and is used to treat spinal chord injury by systemic administration in high doses.

The spinal cord injury was produced under Equithesin anaesthesia (3 ml/kg, i.p.), by making an incision into the right dorsal horn of the T10-11 segments with the use of a sterilized scalpel blade (Sharma et al. Acta Neurochirurgica, Supplement (2003), 86, 399-405)).

10 micrograms of the test compound was administered to the spinal chord. The compounds were administered in water with a total volume of 20 microlitres. The test compounds were administered directly on to the area of injury five minutes after the injury had been produced.

Each test compound was tested on a group of ten rats. The results reported below are averages calculated from the results observed for the ten rats in each group. In a separate group of ten rats the compounds in identical concentration were applied separately on the normal spinal cord over the T10-11 segments 5 min after laminectomy.

The animals were allowed to survive for 12 hours after spinal chord injury and the motoric behaviour was evaluated over this period.

Motoric behaviour and anti-inflammatory effects were evaluated using well known behavioural tests: the angle board method or capacity angle method (Rivlin, A. S., Tator, C. H., J. Neurosurg. 47, 577-581 (1977)), and a Tarlov scale method (Kamencic, H., et al. Faseb J 15, 243-250 (2001) and Tariq, M. et al. J Neurotrauma 15, 239-51 (1998)). Briefly, the Tarlov scale grades motoric ability from normal, grade 6, to complete paraplegia, grade 0. The capacity angle test assesses the animal's ability to remain on an inclined plane. The maximum angle at which an animal can support itself for 5 seconds is the capacity angle; a high value is therefore desired.

This experimental condition is approved by the Institutional Experimental Review Committee, Uppsala, Sweden and Banaras Hindu University, Varanasi, India.

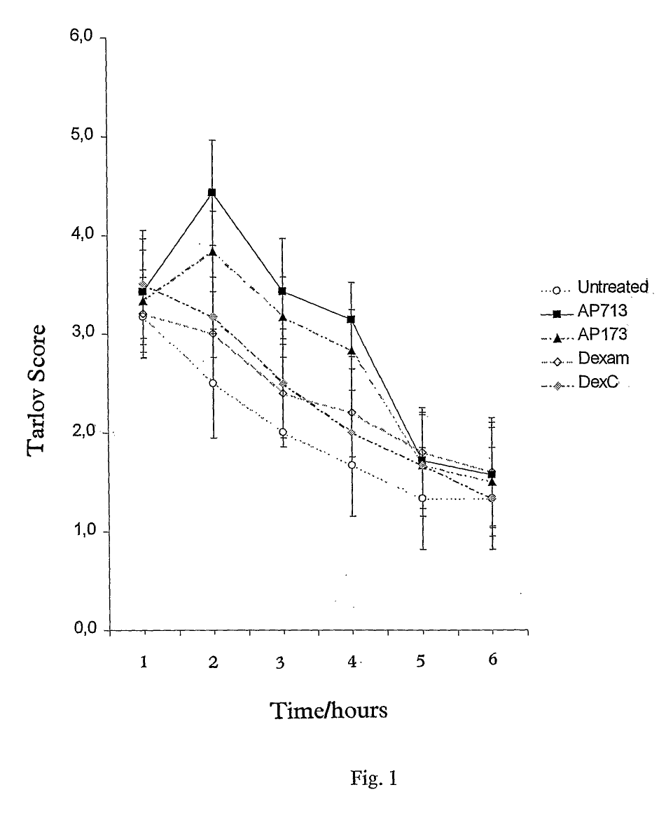

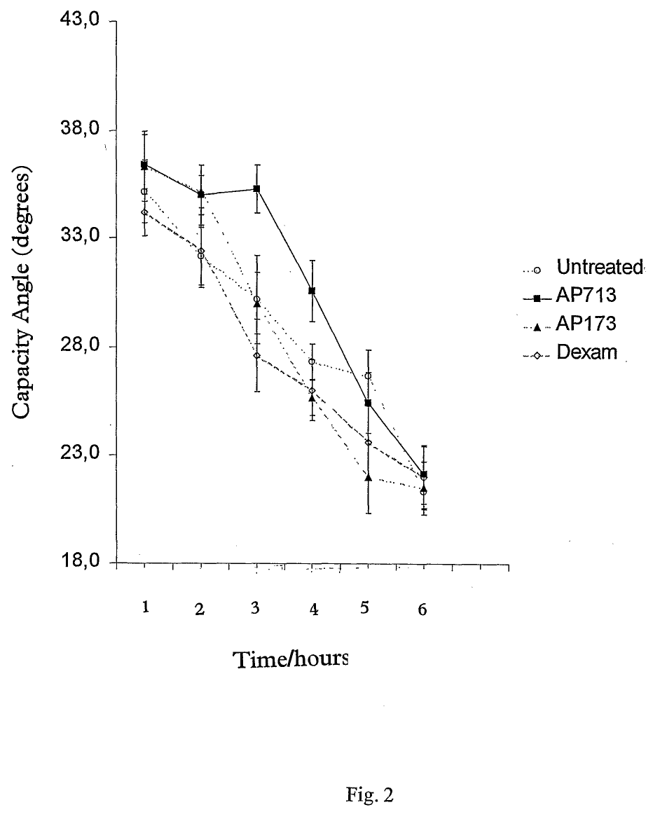

The results from the Tarlov Scale test are shown in FIG. 1 and the results from the Capacity Angle test are shown in FIG. 2.

As illustrated in FIGS. 1 and 2, guanabenz applied topically to a site of injury on the spinal cord had a significant anti-inflammatory effect as well as an improvement of motoric behaviour compared to non-treated or reference compound.

1-28. (canceled)

29. A method of treating neurological damage to the central nervous system in a human or animal, said treatment comprising the topical administration of an effective amount of guanabenz to the site of neurological damage.

30. The method according to claim 29, wherein said neurological damage is a spinal chord injury.

31. The method according to claim 29, wherein said treatment commences within a period of 15 hours after the neurological damage occurs.

32. The method according to claim 29, wherein said treatment comprises a single application of guanabenz.

33. The method according to claim 29, wherein said neurological damage is a spinal chord injury and wherein said guanabenz is applied directly onto the part of the spinal chord that has been damaged.

34. A pharmaceutical composition comprising guanabenz, a glycosaminoglycan or a cellulose derivative as a gel forming agent and water.

35. The composition according to claim 34, wherein said gel forming agent is hyaluronic acid.

36. The composition according to claim 34, wherein said guanabenz is present in an amount of 0.0001-10 weight %, based on the weight of the composition, and said gel forming agent is present in an amount of 0.0001-20 weight %, based on the weight of the composition.

37. The composition of claim 34, wherein said composition comprises one or more ionic surfactants and has a pH of 6 to 9.

38. A pharmaceutical composition comprising guanabenz, wherein said composition is in the form of a non-aqueous ointment.

39. The composition according to claim 38, wherein said guanabenz is present in an amount of 0.0001-2 weight % based on the weight of the composition.

40. The composition of claim 38, wherein said ointment comprises an organic solvent and an ointment emulsifier.
